(19) United States
(12) Patent Application Publication
(10) Pub. No.: US 2023/0149146 A1
(43) Pub. Date: May 18, 2023

(54) AN AUTOMATED CONTROLLED RELEASE DEVICE FOR LIVESTOCK MANAGEMENT AND METHODS OF USE THEREOF

(71) Applicant: CORNELL UNIVERSITY, Ithaca, NY (US)

(72) Inventors: Julio GIORDANO, Ithaca, NY (US); David ERICKSON, Ithaca, NY (US); Yue REN, Ithaca, NY (US); Magdalena Masello SOUZA, Washington, DC (US)

(21) Appl. No.: 17/920,475

(22) PCT Filed: Apr. 27, 2021
(86) PCT No.: PCT/US2021/029431

§ 371 Date: Oct. 21, 2022

Related U.S. Application Data
(60) Provisional application No. 63/016,235, filed on Apr. 27, 2020.

(51) Int. Cl. A61D 19/02

(52) U.S. Cl. CPC A61D 19/027; A61D 19/022; A61M 2205/50

(57) ABSTRACT

An automated controlled device for livestock management and methods of use thereof. The device includes a housing configured to be inserted into a body cavity. One or more reservoirs are configured to be located within the housing and to store a fluid. One or more pumps are coupled to the one or more reservoirs. The one or more pumps are configured to deliver fluid stored in the one or more reservoirs to an area external to the housing during use. A microcontroller is coupled to the one or more pumps. The microcontroller is configured to operate the one or more pumps to deliver a predetermined volume of the fluid stored in the one or more reservoirs to the area external to the housing at a scheduled time

This application claims the benefit of Provisional Patent Application Ser. No. 63/016,235 filed Apr. 27, 2020, which is hereby incorporated by reference in its entirety.

### GOVERNMENT FUNDING

This invention was made with government support under grant number 1014955 awarded by USDA-NIFA. The government has certain rights in the invention.

### FIELD

The present technology relates to an automated controlled release device for livestock management and methods of use thereof.

### BACKGROUND

In cattle farming, cattle reproductive efficiency—the regular production of calves by cows—strongly impacts farm profitability in both dairy and meat enterprises. Low reproductive performance—too few calves per cow per year—causes substantial losses in lifetime productivity and farm profitability because of extended calving intervals, increased culling rates, fewer replacements born, and increased veterinary costs. Management strategies that improve reproductive performance are therefore critical to the profitability of dairy and beef farms.

In modern farm operations, cows are bred using artificial insemination (AI) coordinated with each cow's estrous cycle to maximize the likelihood of fertilization. Many operations now use cow-hormone injections to control ovulation in support of timed artificial insemination (TAI). These protocols assure timely, group insemination and greatly improve pregnancy rates. With TAI there is no need to observe individual cows to detect estrus (which indicates readiness for insemination), and whole groups of cows can be bred synchronously, which simplifies handling. TAI can improve farm profitability and sustainability.

The “Ovsynch” protocol for controlling ovulation using scheduled hormone injections was widely adopted after its advent in the 1990s. Ovsynch revolutionized reproductive management of cattle because it allowed TAI, i.e., breeding cows by appointment. More recently, many similar protocols have been adopted in the US and around the world. Synchronization not only allows a group of cows to be bred at a controlled time without any need to detect estrus (which is labor-intensive, subject to protocol drift, and affected by physiological limitations of cattle, particularly dairy cattle), but improves the success rate of AI and can be used as therapy for cows suffering specific physiological conditions (e.g., anovulation) that dramatically reduce reproductive performance.

All TAI techniques manipulate the cow estrus cycle, a series of endocrine events associated with changes in the cow's reproductive tract. FIG. 1 illustrates the main morphological and hormonal changes throughout the cow estrus cycle. The ovary prepares to release an egg (oocyte) for possible fertilization and development into an embryo; if pregnancy does not occur, the tract prepares for a new round of estrus and ovulation. The cycle is as follows (timings approximate):

Day 1: Ovulation (oocyte release) occurs, and the oocyte can be fertilized. After ovulation, an ovarian structure called the corpus luteum (CL) begins to grow. The CL releases progesterone (P4), needed to maintain pregnancy if fertilization has occurred. P4 is critical for controlling release of gonadotropin hormones, i.e., luteinizing hormone (LH) and follicle-stimulating hormone (FSH). Follicles are ovarian structures that contain one egg each and release hormones.

Days 2-18: The CL matures within the first week of growth, so P4 levels increase dramatically early on and remain high until the last third of the cycle. During this phase, two or three waves of follicle growth and regression occur (a.k.a. follicular waves) in response to the orchestrated release of FSH and LH from the pituitary gland. Around days 16-18, the uterus secretes prostaglandin F2α (PGF), which causes CL regression (a.k.a. luteolysis) and a major decline in P4.

Days 18-23: One follicle becomes dominant. The rest disappear and will not ovulate. As the dominant follicle continues to develop (in a low-P4 environment because of CL regression), it secretes estradiol, which causes estrus (the sexually receptive state). During estrus, estradiol causes the hypothalamus (a part of the brain) to release a surge of gonadotropin releasing hormone (GnRH), which causes the pituitary gland to release a surge of LH, which causes ovulation (release of an oocyte from the dominant follicle), closing the cycle.

The complex sequence of events shown in FIG. 1, including growth and shrinkage of follicles and the CL and the rise and fall of various hormones, dramatizes the fact that the natural estrous cycle can only be crudely approximated by a handful of injections as in current TAI protocols.

The two hormones most commonly used in Ovsynch-type TAI protocols are GnRH and PGF. GnRH is a “releasing hormone,” stimulating the release of other hormones; it triggers primarily release of LH and to a lesser extent of FSH. PGF is the “luteolytic hormone,” i.e., the hormone secreted by the uterus to induce CL regression (a.k.a. luteolysis). Exogenous doses of GnRH and PGF-which must be pulsed and scheduled to achieve desired effects—are the basis of TAI control of the estrous cycle. The simplest TAI protocols feature the following steps, as illustrated in FIG. 2.

1) A first GnRH injection is given to induce a surge of LH, which causes ovulation of an ovarian follicle and thus begins a new follicular wave. 2) Seven days later (typically), PGF is injected to induce luteolysis of one or more CLs on the ovaries (in many cows, a pre-formed CL is present at time of the first GnRH of Ovsynch). 3) 2-3 days later, a second GnRH injection induces ovulation (oocyte release). Ovulation happens ˜24-32 h after GnRH. Zero to 24 hours after GnRH, the cow is artificially inseminated.

More complex injection protocols have been developed, e.g. Double-Ovsynch, Presynch-Ovsynch, and G-6-G, using up to seven separate injections. Also, supplementation with P4 between the initial GnRH injection (step 1) and induction of luteal regression (step 2) is commonly used to improve TAI success rate. P4 is usually delivered by a controlled internal drug release (CIDR) insert, a hormone-impregnated piece of plastic and silicone that provides sustained release of P4 for ≥7 days.

The biggest barrier to adoption and correct administration of state-of-the-art protocols for TAI is the need to administer 3-7 hormone injections (potentially more) over 10-35 days. Many dairy and most beef farms lack the facilities, tools, personnel, and/or frequent access to cows to properly implement such complex protocols. Indeed, issues with labor and animal welfare are intensifying as TAI protocols evolve to include even more numerous, inconveniently timed injections in pursuit of increased efficacy.

In particular, in a TAI protocol, each injection requires that the animal be found, accessed, and in some cases brought to a facility and confined. On a large farm, 4 or 5 workers may be needed on a given day for handling cows for TAI protocols. Smaller farms often lack adequate facilities and cannot afford the trained personnel and expensive, specialized software that supports TAI protocol compliance, while for large operations frequent access to individual cattle is difficult and disruptive of normal cattle behavior. Thus, both large and small farms often struggle to implement TAI in its current forms. Moreover, (1) there is some risk of injury to cow, human, or both during any confined-space procedure such as giving an injection, so it is desirable to minimize the number of such procedures, (2) injection sites blemish beef, causing significant loss of product in the beef industry, and (3) cows are stressed by being repeatedly separated from the herd, placed in a head gate or sorting pen, and injected. Stress lowers dairy cow productivity.

The present technology is directed to overcoming these and other problems in the art.

### SUMMARY

One aspect of the present technology relates to a device. The device includes a housing configured to be inserted into a body cavity. One or more reservoirs are configured to be located within the housing and to store a fluid. One or more pumps are coupled to the one or more reservoirs. The one or more pumps are configured to deliver fluid stored in the one or more reservoirs to an area external to the housing during use. A microcontroller is coupled to the one or more pumps. The microcontroller is configured to operate the one or more pumps to deliver a predetermined volume of the fluid stored in the one or more reservoirs to the area external to the housing at a scheduled time.

Another aspect of the present technology relates to a method for providing automated fluid delivery in a body cavity using the device of the present technology. The device is inserted into the body cavity. Upon insertion, the microcontroller operates the one or more pumps to deliver a predetermined volume of the fluid stored in the one or more reservoirs to the body cavity at the scheduled time.

The present technology advantageously provides a programmable, intravaginal (IVG) device capable of releasing controlled hormone devices at specific times. The present technology automates aspects of cattle breeding to reduce labor, simplify herd management, increase efficacy, and improve animal well-being compared to the elaborate needle-injection methods presently used. The technology may be employed to control cow fertility in preparation for artificial insemination, and be employed for synchronized herd fertility treatment underworking farm conditions.

### BRIEF DESCRIPTION OF THE DRAWINGS

FIG. 1 illustrates the main morphological and hormonal changes throughout the cow estrous cycle.

FIG. 2 illustrates a basic Ovsynch protocol used for timed artificial insemination

FIG. 3 is a front view of an exemplary automated controlled release device for livestock management of the present technology.

FIG. 4A is a front phantom view of the automated controlled release device for livestock management of the present technology.

FIG. 4B is a side phantom view of the automated controlled release device for livestock management of the present technology.

FIG. 5 is a more detailed view of one of the substance delivery mechanisms, reservoirs, and delivery tubing of the automated controlled release device for livestock management of the present technology.

FIG. 6 is a front phantom view of the automated controlled release device for livestock management of the present technology including a flexible silicon bladder.

FIG. 7 is a front phantom view of the automated controlled release device for livestock management of the present technology including an extraction mechanism.

FIG. 8 illustrates an exemplary printed circuit board of the automated controlled release device for livestock management of the present technology.

FIG. 9 illustrates the exemplary automated controlled release device for livestock management of the present technology inserted into a body cavity.

FIG. 10 illustrates blood levels of progesterone after prostaglandin F2α (PGF) or saline treatment. A double intravaginal (IVG) bolus administration of PFG (25 mg per dose) 12 hours apart (PGF25-2X-IVG) produced results indistinguishable from a single dose intramuscular injection of PGF (PGF25-IM). Cows in the saline group (SAL-IVG; negative control) did not present a progesterone decline (as expected).

FIG. 11 illustrates luteinizing hormone concentrations in cows treated with saline solution IVG (SAL-IVG), 100 μg of gonadotropin releasing hormone (GnRH) intramuscular (G100-IM), and 1000 μg of GnRH+10% citric acid IVG (G1000CA-IVG).

FIG. 12 illustrates a single chamber proof-of-concept device for testing IVG PGF delivery.

FIG. 13 illustrates circulating progesterone (P4) concentrations in cows treated with prostaglandin F2α (PGF) intravaginal (IVG) with the device shown in FIG. 12 (e-Synch PGF), saline with the device shown in FIG. 12 (e-Synch-Sal), IVG delivery through catheter (IVG-Cath-PGF), and intramuscular injection (IM-PGF). 2-bolus administration with the device shown in FIG. 12 produces similar results to intramuscular and catheter administration.

FIG. 14A illustrates representative delivery curves of 4 pumps showing amount (g) of distilled water delivered over a 300 s period.

FIG. 14B is a Bland-Altman plot, with the differences between target and delivered dose plotted against target dose. Pumps (n=4) were programmed to release different target volumes (0.1, 0.2, 0.5, 1.0, and 2.0 g) in 4 replicates (n=80 doses). The mean difference (−0.005 g) is represented by the solid line and the 95% confidence limits by the dashed lines, whereas the solid grey line represents the zero-bias line (i.e., 0 g).

FIG. 14C is a graph depicting the average amount (g) delivered by two IVG devices programmed to release four 2.0 g doses (target dose; dashed line) following a scheme of one dose every 12 hours.

FIG. 15 illustrates circulating concentrations of progesterone (P4) from 0 to 72 h after application of treatments. Lactating Holstein cows with at least 1 corpus luteum >15 mm in diameter were randomly allocated to 1 of 3 treatments: IM-PGF (n=6); two 25 mg intramuscular doses of PGF 24 h apart, DEV-PGF (n=6); four 25 mg doses of PGF released automatically by the IVG device every 12 h, and DEV-CTL (n=4); insertion of an empty IVG device (placebo control). Values are presented as LSM±SEM. *Progesterone differed from 24 to 72 h as cows in IM-PGF and DEV-PGF had lesser P4 than cows in DEV-CTL.

FIG. 16 illustrates individual plasma progesterone profiles from 0 to 72 h after treatment for (A) cows in DEV-PGF, (B) cows in IM-PGF, and (C) cows in DEV-CTRL.

### DETAILED DESCRIPTION

One aspect of the present technology relates to a device. The device includes a housing configured to be inserted into a body cavity. One or more reservoirs are configured to be located within the housing and to store a fluid. One or more pumps are coupled to the one or more reservoirs. The one or more pumps are configured to deliver fluid stored in the one or more reservoirs to an area external to the housing during use. A microcontroller is coupled to the one or more pumps. The microcontroller is configured to operate the one or more pumps to deliver a predetermined volume of the fluid stored in the one or more reservoirs to the area external to the housing at a scheduled time.

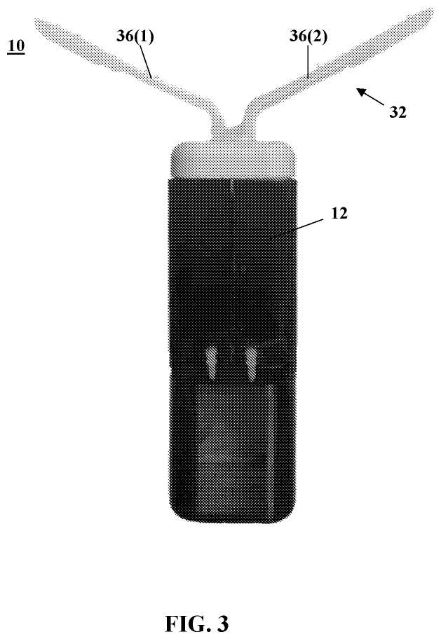

FIGS. 3-9 illustrate an automated controlled release device 10 for livestock management. Device 10 includes housing 12, substance delivery mechanism 14, reservoirs 16 luer lock connections 18, delivery tubing 20, printed circuit board (PCB) 22, battery 24, sensors 26(1)-26(n), and microcontroller 28, although device 10 could include other types and/or numbers of elements or components in other combinations. The present technology advantageously provides a programmable, intravaginal (IVG) device capable of releasing controlled hormone devices at specific times. Further, the device incorporates design features (e.g., smaller pumps, substance reservoirs with no plunger, shorter substance reservoirs, silicone bladder, fewer batteries, smaller batteries, and shorter tubing) that enable shortening device 10, which is critical to enable maximal retention of the device when inserted in a body cavity. The present technology, in one example, enables TAI with automated delivery of hormones. TAI with automated delivery of hormones can decrease labor, lower animal stress, and likely increase efficacy. The present technology enables TAI automation to eliminate missed injections (protocol noncompliance) and reduce labor needs. The present technology further enables (1) unconstrained, biomimetic dosing schedules and (2) adaptive dose sizing and/or scheduling in response to sensed conditions (e.g., body position, physiological indicators).

Referring now more specifically to FIG. 3, housing 12 has a smooth outer surface to facilitate insertion into and removal from the body cavity, and to minimize reaction by the body cavity, as the device is a foreign body to the subject. For example, housing 12 is configured to be inserted intravaginally to be employed as an IVG device as illustrated in FIG. 9. Housing 12 is formed of a plastic material, although other materials may be employed. In one example, a robust, carbon-fiber-filled thermoplastic may be used to increase durability. In one example, housing 12 may be formed using 3-D printing.

In this example, housing 12 includes first and second outer plastic housing components (front 42 and back 44), although housing 12 may have other configurations. Housing 12 is divided in two pieces, as described below, to enable substance delivery and avoid malfunction due to entrance of moisture and mucus produced in body cavities.

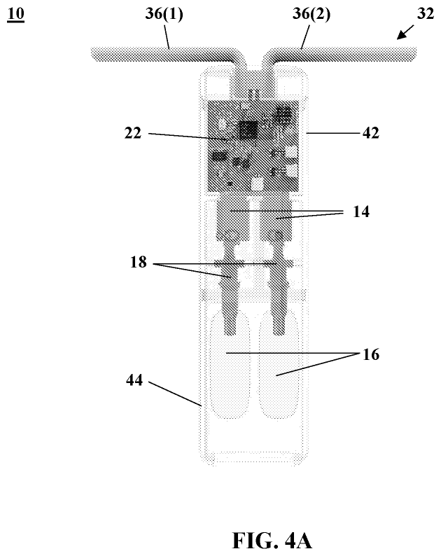

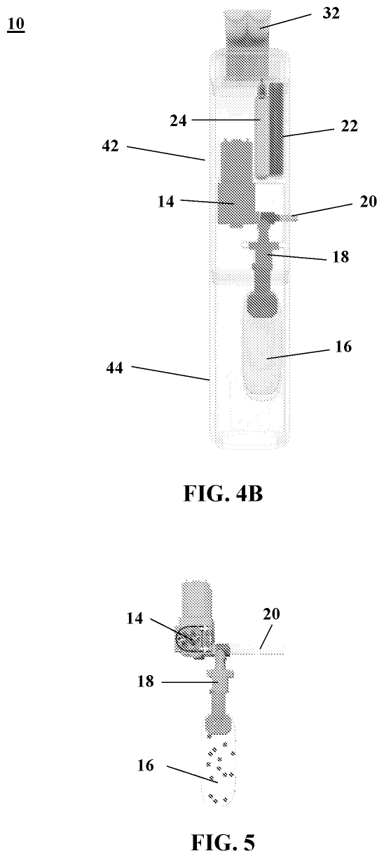

Referring now more specifically to FIGS. 4A and 4B, the first or front component 42 of housing 12 houses substance delivery mechanism 14, PCB 22, and battery 24. The front component 42 also serves as an anchor for luer lock connections 18. The front component 42 is completely sealed and cannot be disassembled to avoid entrance of moisture and mucus. Housing 12 includes separate components to avoid internal generation of vacuum conditions in the front component due to displacement of substance from reservoirs 16 in a fully sealed device, as described in further detail below. Vacuum generated by the negative pressure substance displacement mechanism forces moisture and mucus into the device, which corrodes electronics and prevents function. Thus, locating reservoirs 16 in the back component and completely sealing front component of housing 12 avoids moisture being drawn into the front component of housing 12 to protect the components located therein. The front component 42 of housing 12 includes a connection mechanism (e.g., screw cap) to connect to the back component 44 of housing 12.

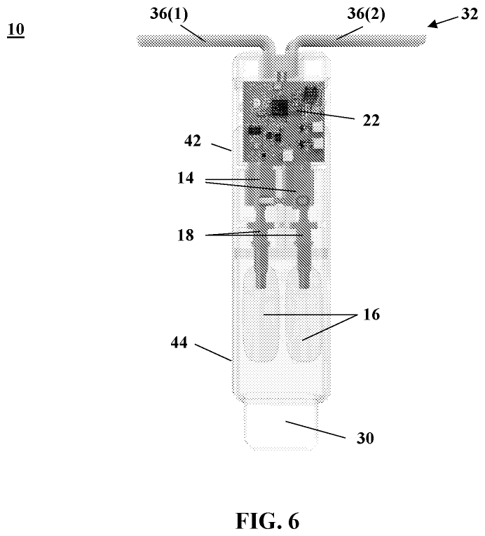

The second or back component 44 of housing 12 provides a hard plastic case that, in one example, has an attached silicone bladder 30, as shown in FIG. 6. The back component 44 of housing 12 is configured with a size and shape to house substance reservoirs 16 when fully loaded. In one example, the back component 44 is configured to removably receive the reservoirs 16. The back component 44 of housing 12 is attached to the front component 42 for operation of device 10, as described above. Flexible silicone bladder 30 enables delivery by filling space created by displacement of substance from reservoirs 16 and a completely sealed device during operation. Silicone bladder 30 enables substance delivery by molding inwards into the back of housing 12 in response to negative pressure generated by displacement of substance in reservoirs 16. Lack of resistance to inward molding by flexible silicone bladder 30 enables substance delivery by negative pressure system by reducing the power required by substance delivery mechanism 14 to extract substance from substance reservoirs 16. Silicone bladder 30 enables device 10 to be fully sealed, thus preventing entrance of moisture and mucus into device 10.

Referring again to FIGS. 3, 4A, and 4B, external housing 12 also includes retention mechanism 32. The retention mechanism 32 is configured to retain the housing in the body cavity during use. In one example, retention mechanism 32 comprises a pair of opposing members 36(1) and 36(2) configured to be folded into the housing and to provide an external pressure away from housing 12 against the body cavity during use, although in another example to pairs of opposing members could be employed. In one example, opposing members 36(1) and 36(2) are a pair of semi-rigid wings, although other configurations may be employed. In this example, opposing members 36(1) and 36(2) of retention mechanism 32 are foldable so that they can be folded during insertion of device 10 with an applicator and released when inserted into the body cavity to exert pressure against the body cavity to remain in place during use. In one example, the retention mechanism 32 is formed of plastic and silicone, although other semi-rigid materials may be employed. In one example, retention mechanism 32 is part of the front component of housing 12.

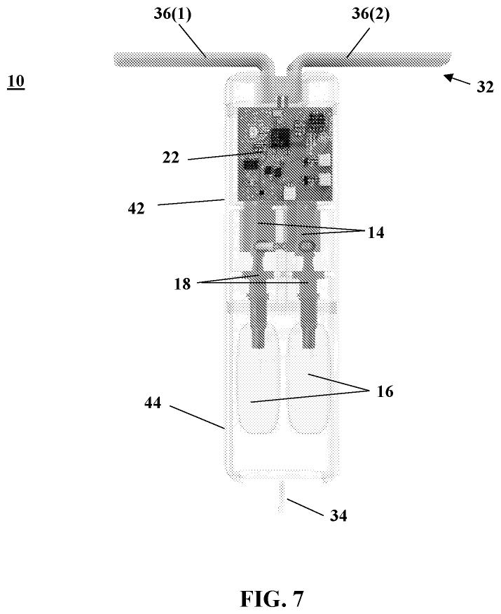

Referring now more specifically to FIG. 7, in one example, housing 12 further comprises extraction mechanism 34 to allow for removal of housing 12 from the body cavity. In one example, extraction mechanism includes a coil configured to extend from housing 12 outside of the body cavity during use. Extraction mechanism 34 is formed as a semi-flexible plastic coil and is of sufficient length to extend beyond an opening of the body cavity to enable manual extraction pulling for removal from the body cavity. The coil can also be used to balance the internal pressure change in device 10 during operation. In another example, extraction mechanism 34 may be a string to allow for removal of device 10.

Referring again to FIGS. 4A, 4B, and 5, substance delivery mechanisms 14 includes one or more pumps coupled to reservoirs 16 by the luer lock connections 18, although other mechanical coupling mechanisms may be employed. The one or more pumps are coupled to reservoirs 16 and are configured to deliver fluid stored in reservoirs 16 to an area external to housing 12 through delivery tubing 20 during use. In one example, device 10 includes a pump for each of the reservoirs employed. Although two substance delivery mechanisms 14 are shown in FIG. 4A, it is to be understood that any number of substance delivery mechanisms 14 could be employed.

In one example, the one or more pumps used for substance delivery mechanisms 14 are negative pressure pumps. For example, miniaturized osmotic pumps may be employed to provide delivery of substance from reservoirs 16. Utilizing negative pressure pumps for substance delivery mechanisms 14 enables larger amounts of substance delivery, while limiting the size of device 10. Body cavities (e.g., vagina) limit the size of device 10 and reducing size is critical for introduction and removal from the subject, as well as subject comfort during treatment. Positive pressure mechanisms such as those that use plungers actuated by solenoids or springs occupy large amounts of space. Positive pressure systems including rigid plastic reservoirs with plungers actuated by solenoids would result in larger than desired devices (i.e., larger than body cavity for insertion). Gas-cell systems used to generate positive pressure to displace a plunger contained within the substance reservoir are hard to control, which makes it difficult to control the amount of substance released from reservoirs and need to be replaced after each use.

Unlike positive pressure mechanisms, negative pressure mechanisms (i.e., mechanisms that extract substance from reservoirs 16 by creating negative pressure or vacuum) with miniaturized osmotic pumps enable size reduction for device 10 and a ratio of delivery mechanism to reservoir size ratio. Thus, the use of miniaturized osmotic pumps enables maximizing the size of substance reservoirs 16 within constrains of the total size of device 10. Negative pressure systems, such as those that use osmotic pumps, further advantageously eliminate the need for outlet valves to prevent leakage of substance from substance reservoirs 16. Leakage of substance from reservoirs 16 can exert a physiological response at inappropriate times, which leads to treatment failure. Leakage of substance from reservoirs 16 also reduces amount of substance for future needed treatments.

Substance delivery mechanism 14 includes miniaturized osmotic pumps in order to generate sufficient strength to enable displacement of the substance from reservoirs 16 and through delivery tubing 20 into the subject body cavity. Substance delivery mechanism 14 has enough strength to displace substance from nozzles into the body cavity of the subject even when mucus or other fluids have accumulated and block the nozzle opening. Miniaturized osmotic pumps have enough strength to displace fluid through mucus or contents of the body cavity that may block device nozzles.

Substance delivery mechanisms 14 including actuation systems based on miniaturized osmotic pumps further enables re-use of substance delivery mechanisms 14 and device components. No parts need to be replaced or serviced in between uses. Additionally, miniaturized osmotic pumps have low energy consumption, which enable use of smaller batteries for battery 24, and thus reduces the overall size of device 10. Minimizing the size of device 10, particularly in length provides improved retention of device 10. Limiting length of device 10 advantageously avoids device 10 being expelled from the body cavity, e.g., a vagina, by peristalsis. Retention of device 10 within the body cavity is required for treatment success, as substances must be absorbed by contact with the mucosal membrane of the body cavity, for example.

In this example, device 10 includes substance delivery mechanism 14 that includes a single miniaturized osmotic pump for each of substance reservoirs 16. This configuration enables completely independent release of a substance from each of individual substance reservoirs 16 employed. Independent release of a substance from each of the reservoirs 16 enables treatment regimens that combine different substances delivered at desired time points, in desired amounts, and at desired rates without affecting the release of other substances from other ones of reservoirs 16. Unrestricted substance release enabled by individual pumps enables unrestricted treatment options to optimize biological response to the substance delivered and the subject performance.

In this example, substance delivery mechanisms 14 includes one or more pumps configured to release the fluid from reservoirs 16 in a surge release or a sustained release. Unlike positive pressure system actuated by other means, negative pressure systems based on miniaturized osmotic pumps enable release of small and large amounts at desired at rates that enable mimicry of surge (i.e., equal to bolus injection by other routes) or sustained release of a substance. For example, substance delivery mechanisms 14 enable release of several milliliters or milligrams in minutes or a few milliliters or milligrams in days. Mimicry of surge and sustained release pattern enables mimicry of subject physiological response caused by or in response to the substance released. For example, it is desired to release certain substances such as gonadotropin releasing hormone (GnRH), prostaglandin F2α (PGF), luteinizing hormone (LH), follicle stimulating hormone (FSH), equine chorionic gonadotropin (eCG), estradiol (E2), organic acids, saline or water to cause or as a surge whereas sustained release is desired for other substances such as progesterone, as described in further detail below. Although certain substances are described it is to be understood that the present technology could be utilized with other liquid or semi-liquid substances to provide other treatment protocols in an automated and controlled manner.

Device 10 further includes reservoirs 16 configured to be located within housing 12 and to store a fluid. Device 10 stores and delivers substances in liquid and semiliquid form. Substances delivered by device 10 include substances used to control one or more biological functions in the subject. Control of one or more biological functions individually or in combination enable management intervention (e.g., insemination at detected estrus, timed artificial insemination, superovulation, ovum pick up). By way of example, two silicone hormone reservoirs 16 that hold approximately 5 mL of fluid may be employed, although other types and/or numbers of reservoirs may be employed based on the desired treatment regimen. In another example, 2-4 reservoirs 16 of 6-8 mL each may be employed. In one example, at least two reservoirs are needed to deliver the minimum number of different hormones to synchronize ovulation, i.e., GnRH and PGF. A third reservoir may be employed for P4 and a fourth reservoir may be employed to provide total greater volume for a hormone (e.g., GnRH or PGF), to add a fourth hormone to a protocol, or to add hormone absorption enhancer. In other examples, a plurality of reservoirs 16 may be employed that are each independently accessed by one of the one more pumps of substance delivery mechanisms 14 to allow independent fluid delivery from each of the plurality of reservoirs 16 during use.

In one example, reservoirs 16 are formed of silicone, although other suitable materials may be employed. The use of silicone provides reservoirs 16 that are flexible to enable substance delivery using a negative pressure system, as well as allowing for expanded substance storage capacity. The use of silicone for reservoirs 16 further enables the use of miniaturized osmotic pumps for substance delivery mechanisms 14 to displace substance from reservoirs 16 by negative pressure to deliver a desired amount at a specific rate.

As described above, substance reservoirs 16 are separately located in the back component of housing 12. Reservoirs 16 are coupled to substance delivery mechanisms 14, which are located in the front component of housing 12, through a connection system which enables reuse of substance delivery mechanisms 14 and replacement of substance reservoirs 16. Reuse of substance delivery mechanisms 16 reduces overall cost of device 10. Replacement of substance reservoirs 16, which are removably inserted into the back component of housing 12, enables reuse of device 10, reduces costs, and allows for easy refilling of reservoirs 16 for prolonged treatments.

In one example, substance reservoirs 16 are single use and may be removably inserted into device 10. Housing 12 may include a hatch that allows for quick exchange of pre-filler reservoirs 16. Substance reservoirs 16 are plugged in every time a substance, such as a hormone, must be delivered which enables different combinations of substance type and delivery amounts to be employed. Enabling different combination of type of substances and amounts of each substances at each use enables use of device 10 for different treatments, such as the treatments described herein. Single use substance reservoirs 16 further reduces cleaning time before reusing reusable components and minimizes risk of infection.

Luer lock connections 18 are anchored in the front component of device 10 and enable connection of substance reservoirs 16 to negative pressure substance delivery mechanisms 14. Once substance reservoirs 16 are connected to luer lock connections 18, substance can be driven out of reservoirs 16 by negative pressure exerted by the delivery pumps of substance delivery mechanisms 14.

Delivery tubing 20 is coupled to substance delivery mechanisms 14, such as one or more pumps, to allow fluid drawn from reservoirs 16 to be delivered to an area external to housing 12. For example, housing 10 may be positioned intravaginally to allow delivery of fluids to the vaginal cavity during use for TAI as described herein.

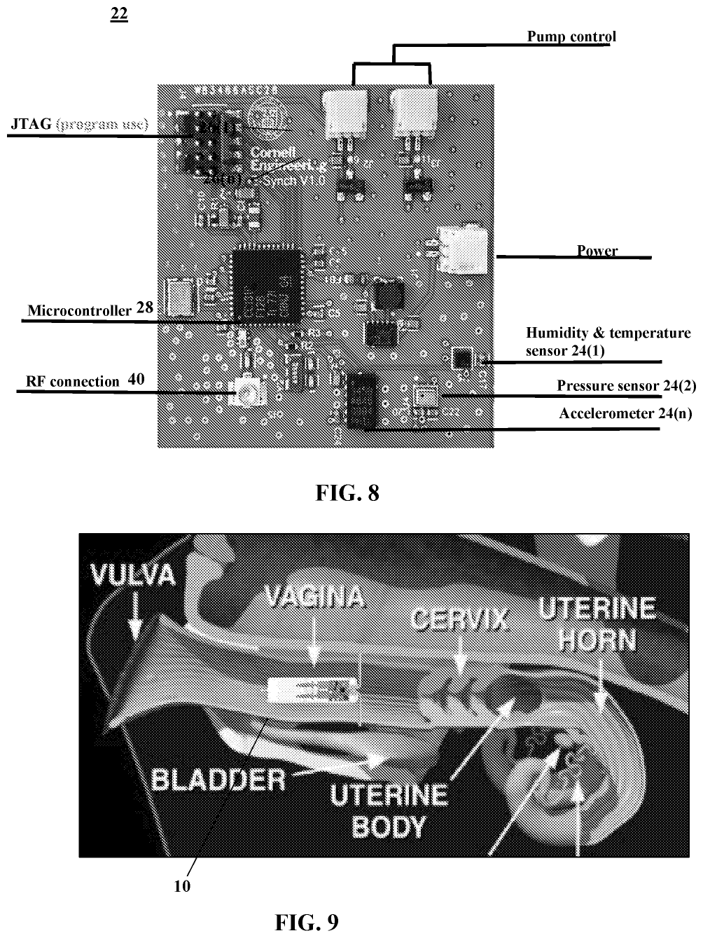

PCB 22 is located within housing 12 and supports the electronic components of device 10 including microcontroller 28, as described below. In one example, the PCB is a four-layer PCB having dimensions of 34 mm×34 mm, although other types and/or sizes of PCB may be employed. In one example, PCB 22 has a diameter of about 3.5 cm and a thickness of about 1 cm, although other dimensions may be employed. PCB 22 is sized to provide an overall compact design for device 10 for insertion into a body cavity. Referring now more specifically to FIG. 8, PCB 22 also supports the electronics of device 10 including microcontroller 28 and sensors 26(1)-26(n), although PCB 22 may include other types and/or numbers of electronics located thereon.

Referring again to FIG. 4B, battery 24 is located within housing 12 and is electrically coupled to and used to power one or more components of device 10, such as substance delivery mechanisms 14, sensors 24(1)-24(n) and microcontroller 28, for example. In one example, batter 24 is a 3.7 V 450 mAh rechargeable battery, although other types of batteries may be employed. Most of the time, the electronics of device 10 will be in a standby mode consuming 0.1 μA of electricity. Driving substance delivery mechanisms 14, such as one or more pumps, will require 35 mA in some examples. RF communication, as described below, will take 5.4 mA for receiving and 13.4 mA for transmitting, by way of example. A rechargeable 450 mAh Lip battery will allow device 10 to operate for approximately 35 days.

Sensors 26(1)-26(n) are coupled to housing 12 and are configured to determine one or more items of information regarding the environment and/or the movement of device 10. In one example, one or more of sensors 26(1)-26(n) may be located on PCB 22. For example, sensors 26(1)-26(n) may include temperature, moisture, and barometric pressure sensors to provide information regarding placement of device 10 in the body cavity. In one example, a barometric pressure sensor (BMP280, Bosch Sensortec), humidity and temperature digital sensors (HDC2010, Texas Instruments™) and accelerometer (ADXL345, Analog Devices) are integrated on PCB 22. Sensors 26(1)-26(n) are coupled to microcontroller 28 to provide data to microcontroller 28 regarding placement of device 10. Placement of device 10 determined by the items of data from sensors 26(1)-26(n) may be employed by microcontroller 28 to control substance delivery. For example, microcontroller 28 may only deliver substance if a determination is made that device 10 is placed within the body cavity.

In one example, at least one of sensors 26(1)-26(n) is an accelerometer. Accelerometer data may be employed by microcontroller 28 to determine subject posture and positioning of device 10 within the body cavity. In one example, a pedometer algorithm is employed for motion monitoring such that x-y-z acceleration is used to extract step information, compensating for rotation of device 10 in the body cavity. Microcontroller 28 may adjust delivery of the substance based on subject posture and positioning of device 10 within the body cavity. For example, as described below, microcontroller 28 may be configured to deliver substance when subject is certain position (e.g., standing, laying) and device nozzles (i.e., elbow shaped nozzles) are in a certain position (e.g., facing down in contact with mucosa of body cavity) as determined by accelerometer data. Delivery while the subject is certain positions avoids backflow of substance from body cavity by gravity (i.e., when laying down). Delivery while the nozzles of device 10 are facing down only enables enhanced substance absorption by maximizing contact with mucosa of the body cavity. Preventing backflow of delivered substance and contact with mucosal surface of the body cavity maximizes substance absorption increasing treatment efficacy.

Sensors 26(1)-26(n) may further include temperature, moisture, or barometric pressure sensors to enable substance delivery tailored to physiological, behavioral, and performance conditions of the subject and to allow modification of substance delivery schemes to optimize substance biological response and efficacy of treatments based on delivery of one or more substances. For example, accelerometer and temperature sensors enable detection of estrus behavior. Temperature data, for example, aids in determining position in the body cavity, such as a vagina, reaction of the body cavity to device 10 (e.g., infection based on elevated temperature), and device internal temperature (relevant to operation of electronics and pumps), and may aid with estrus detection (vaginal temperature varies around estrus and ovulation). Expression of estrus by the subject enables use of different amounts of substance (i.e., dose) delivered for individual treatments or total amount of substance during treatment that enables management action, substance delivery frequency, and substance delivery rate (i.e., amount of substance delivered per unit of time). Expression of estrus enables continuation or discontinuation of substance release by device.

Microcontroller 28 is located on PCB 22. In one example, microcontroller 28 is the CC1310 SimpleLink™ Ultra-Low-Power Sub-1 GHz Wireless MCU (Texas Instruments™) although other types and/or numbers of microcontrollers or other computing devices may be employed. In one example, microcontroller 28 is integrated with sensor controller engine 38 for control of sensors 26(1)-26(n) and radiofrequency (RF) core 40 for long range wireless communication, although microcontroller 28 may include other elements, such as other communication interfaces. The sensor controller engine 38 controls peripherals autonomously from the rest of the system, which allows the microcontroller 28 to operate in a low power consumption mode.

The RF core 40 autonomously handles the radio protocol with a wide range of modulation formats and data rate from 625 bps to 4 Mbps, by way of example. In one example, a long-range mode at 914 Mhz, 625 bps with −124 dBm receiver sensitivity and 2-FSK modulation is employed for communication, although other communication protocols may be employed. In this long-range mode, the signal can travel up to 7.5 meters with an antenna when located in the body cavity, such as a vagina, and more than 50 meters with the antenna exposed outside. In one example, the antenna may be located on extraction mechanism 34 to be positioned outside of the body cavity during use. In one example, RF core 40 allows for communication with a base station, such as a LaunchPad in serial with a Beaglone Black, for example, to receive data from device 10 for upload to the cloud. Although wireless interaction is described, it is to be understand that microcontroller 28 can be configured to operate device 10 without wireless communication. The wireless data link provides additional features, such as real-time data and the ability to modify the delivery protocol.

RF core 40 or other communication interface associated with microcontroller 28 allows device 10 to integrate with the ongoing explosion in digital farming, the uses of automation, big data, analytics, and specialized machinery that are revolutionizing agriculture. Remote sensors and other devices for animal management to increase profitability and animal well-being are proliferating, including in the cattle industry, and are increasingly managed by specialized software. In one example, data from device 10 is manageable by a device-specific application. Further, device 10 may interact with other software applications employed by farms. In one example, device 10 may interact with an application that includes submenus for selecting industry standard hormone protocols and for entering user-defined treatments, by way of example. The latter will facilitate treatment of cows, singly or in groups, based on form needs, physiological status, or prescriptive treatment for individual cows to optimize reproductive performance. Treatment schedules for specific protocols can be uploaded to microcontroller 28 of device 10 through the application prior to insertion into the body cavity. Once uploaded, device 10 can operate autonomously without need for external intervention. The wireless communication system can be used to download temperature and activity data on demand or as needed.

An exemplary operation of device 10 will now be described with respect to FIGS. 3-9. In one example, device 10 is used for synchronizing ovulation with bolus administration of GnRH and PGF. First, reservoirs 16 are filled with a substance for delivery into the body cavity. In one example, two reservoirs 16 are employed as shown in FIGS. 4 and 5 and are separate filled with GnRH and PGF, although other fluids or semi-fluids may be employed, including other types of hormones. In other examples, additional reservoirs could be employed to provide other types of treatment protocols. Filled reservoirs 16 may be inserted into housing 12 and coupled to substance delivery mechanisms 14 through the luer lock connections 18.

Once reservoirs 16 are installed in housing 12, device 10 inserted in the body cavity. By way of example, device 10 may be inserted into the vagina of a cow to provide TAI, as shown in FIG. 9, although other uses in other body cavities of other animals may be contemplated. Next, device 10 provides controlled, timed, automatic release of the substances from reservoirs 16 into the body cavity. In one example, controlled release is accomplished by setting the voltage of the GPIO pins in microcontroller 28. Microcontroller 28 is programmed in C language to deliver a preprogrammed volume of a hormone at a scheduled time. In example, two GPIO pins are routed and programmed to switch the ON/OFF of N-Channel MOSFETs (DMN65D8L, DIODES, Inc), which control the power supply from battery 24 for each substance delivery mechanism 14, such as a peristaltic pump. The dosing volume is controlled by the ON/OFF time with high linearity, and the delivery speed for each substance delivery mechanism 14 is calibrated in the in vitro test. Once powered on, device 10 starts to count down the sleeping time which is preprogrammed according to the delivery schedule with current rate of 0.7 uA (without sensors 26(1)-26(2)). Upon waking up, microcontroller 28 starts to drive the preprogrammed pump for a programmed time and consumes 40 mA current. Once finished, microcontroller 28 returns to sleep mode and count down again for the next release.

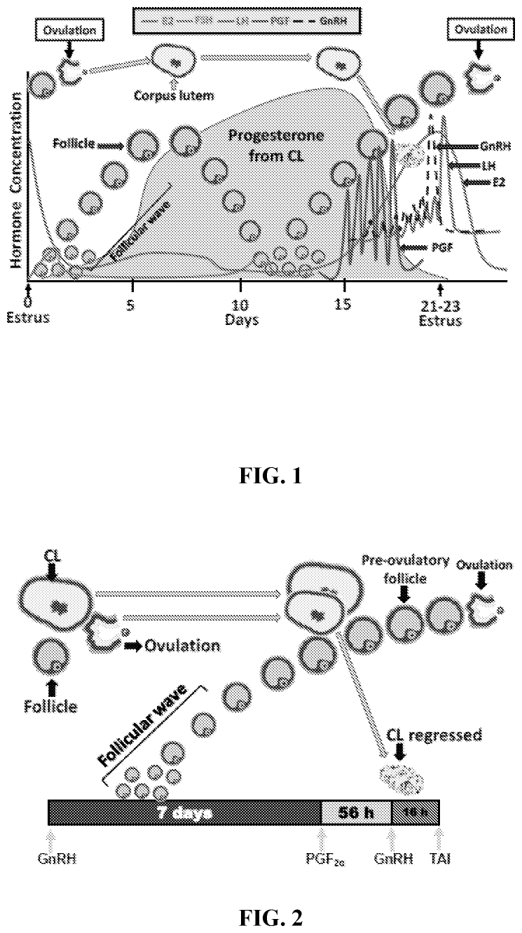

In one example, operation of substance delivery mechanisms 14, such as one or more pumps, is adjusted by microcontroller 28 based on estrous behavior. Estrous behavior is determined by physiological data obtained by sensors 26(1)-26(n). Estrus detection is not required for TAI protocols, but estrous behavior can be used by device 10 to modify treatments in real-time (e.g., change dosage or timing in cows in estrus and complete the TAI protocol, such as illustrated in FIG. 1).

In one example, device 10 may be employed for investigation of arbitrarily complex, biomimetic hormone administration schedules, which are out of the question for injection-based TAI. Device 10 enables an entirely new avenue of managed-fertility research.

Another aspect of the present technology relates to a method for providing automated fluid delivery in a body cavity using the device of the present technology. The device is inserted into the body cavity. Upon insertion, the microcontroller operates the one or more pumps to deliver a predetermined volume of the fluid stored in the one or more reservoirs to the body cavity at the scheduled time.

### EXAMPLES

### Example 1—Efficacy of IVG Delivery

The efficacy of IVG delivery, as opposed to needle injection, of the two most important hormones for synchronization, i.e., gonadotropin-releasing hormone (GnRH) and prostaglandin F_{2α} (PGF) was verified.

Unlike other hormones, such as P4, which requires slow and sustained release, GnRH and PGF must quickly reach elevated serum levels to elicit a physiological response. Rapid delivery (i.e., over seconds or minutes rather than hours or days) of these hormones is therefore required, whether by injection or other means. Further, the hormone surges must occur according to a strict timeline. All current commercial TAI methods deliver hormones by intramuscular or subcutaneous needle injection. The present technology provides for electronically-controlled IVG bolus delivery, not used in any prior TAI protocol. Thus, IVG delivery has been verified.

The vagina has favorable attributes for hormone delivery: it is easily accessible, it offers a sheltered environment for a delivery device of the size required, its mucosa is densely vascularized and highly permeable to low-molecular-weight molecules, and substances absorbed through its walls avoid the first-pass effect (loss of a fraction of an ingested pharmaceutical in passage through the gut wall and liver). However, data for the efficacy of IVG hormone delivery have been sparse. Two studies tested the ability of IVG-delivered PGF to induce luteolysis in cattle, but were inconclusive because they observed few cows or a short time interval (36 h). There has been substantial evidence for release of LH and ovulation after IVG delivery of GnRH in non-cattle species and reports of successful induction of LH release and ovulation after intrauterine delivery of GnRH in cattle.

Catheters were employed to deliver IVG hormones in lactating Holstein cows. Results show that IVG-delivered PGF and GnRH are absorbed and have the desired physiological effects.

### PGF Experiments

In a first study, regression of the corpus luteum (“CL regression”) and plasma P4 concentrations after IVG delivery of 25, 50, or 125 mg PGF (Dinoprost) or of saline 7.5 days after induction of ovulation by Ovsynch injections was evaluated. A positive control group received 25 mg of PGF im (i.e., by intramuscular injection), enabling a comparison of outcomes with im PGF to those with IVG PGF. A single bolus of IVG PGF was found to be less effectual than im PGF, i.e., did not guarantee CL regression, but in a follow-up experiment, 100% of cows given two 25 mg boluses of IVG PGF 12 hours apart experienced complete CL regression as illustrated in Table 1 set forth below:

TABLE 1
Frequency of complete luteal regression and mean concentrations of progesterone
at 60 and 72 h after treatment for cows in two experiments [29].

| | Time after treatment | | | | | |
|---|---|---|---|---|---|---|
| | 60 h | | | 72 h | | |
| Treatment¹ | CLR % (n/n) | | P4 (ng/mL) | CLR % (n/n) | | P4 (ng/mL) |
| SAL-IVG | 0^{a} | (0/13) | 6.26 ± 0.54^{a} | 0^{a} | (0/13) | 5.99 ± 0.47^{a} |
| PGF25-IM | 86^{b} | (12/14) | 0.67 ± 0.23^{bc} | 86^{b} | (12/14) | 0.57 ± 0.24^{c} |
| PGF25-IVG | 42^{b} | (5/12) | 1.74 ± 0.50^{b} | 42^{b} | (5/12) | 2.24 ± 0.64^{b} |
| PGF25-2X-IVG | 100^{b} | (6/6) | 0.19 ± 0.04^{c} | 100^{b} | (6/6) | 0.21 ± 0.07^{c} |

¹Cows received the following treatments 7.5 d after 2nd GnRH of Ovsynch: SAL-IVG = single IVG bolus 5 mL saline. PGF25-IM = single im bolus 25 mg PGF, PGF25-IVG = single IVG bolus 25 mg PGF, PGF25-2X-IVG = two IVG boluses 25 mg PGF 12 h apart. CLR = complete luteal regression (P4 < 1 ng/mL). CLR and mean P4 were affected by treatment (P < 0.001).
^{abc}Different superscripts within column differ statistically (P < 0.05).

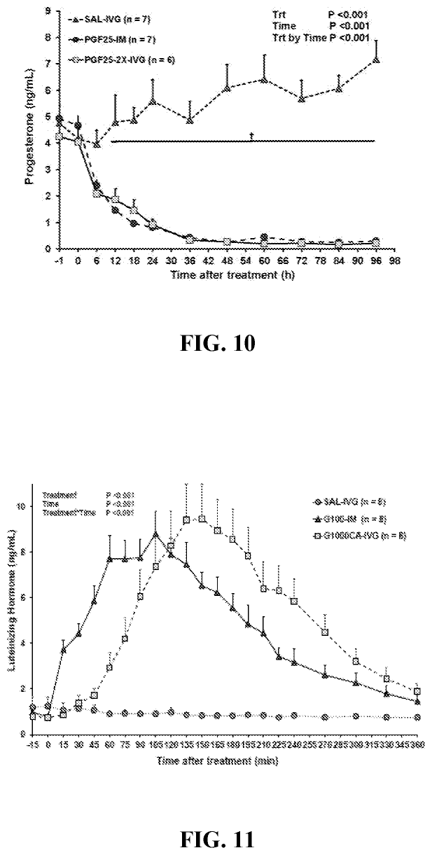

Another positive aspect of the two IVG boluses was the very low P4 concentration after treatment: plasma P4 in these cows was similar to that in cows given intramuscular PGF as illustrated in FIG. 10. Low plasma P4 after the induction of luteolysis (i.e., with 48-72 hours) is critical to TAI success, as mounting evidence indicates that P4 concentrations in the 0.4-0.5 ng/mL range at induction of ovulation with GnRH are optimal for fertility of cattle.

The PGF experiments' ability to show equal luteal regression with im and IVG administration was limited by small N. Data from a larger, more recent experiment supports non-inferiority for IVG compared to im delivery of PGF to induce luteal regression. These data are more appropriate for CL regression (a binary outcome) as the study included approximately 350 cows. Cows that received two 25 mg doses of PGF (Dinoprost) 12 hours apart had similar CL regression rate, estrus expression rate, and ovulatory response after GnRH treatment to cows treated with the standard dose of PGF im (25 mg of Dinoprost) as shown in Table 2 below.

TABLE 2
Physiological outcomes for cows treated with PGF im or IVG [33].

| | Treatment¹ | | |
|---|---|---|---|
| Outcome | IM (n = 169) | IVG (n = 179) | P-value |
| CL regression², % | 81.1 | 81.0 | 0.48 |
| P4 (ng/mL) at 48 h | 0.4 ± 0.1 | 0.5 ± 0.1 | 0.41 |
| P4 (ng/mL) at 72 h | 0.4 ± 0.1 | 0.6 ± 0.1 | 0.18 |
| Estrus after PGF³, % | 47.0 | 51.1 | 0.46 |
| Ovulation⁴, % | 88.0 | 84.1 | 0.30 |

¹Seven days after induction of ovulation with GnRH cows received 25 mg of PGF im or two 25 mg doses of PGF 12 h apart. Dinoprost used for both groups.
²Progesterone reduction from >1 ng/mL at time of treatment to <0.5 ng/mL 72 h after treatment.
³Estrus detected based on increased physical activity within 72 h of treatment.
⁴Ovulation in response to a GnRH treatment given within 72 h of PGF treatment.

In sum, the PGF experiments confirmed the feasibility of inducing complete CL regression through IVG delivery of PGF, an essential aspect of estrous cycle manipulation for TAI. Also, the results confirmed that two IVG boluses of PGF 12 hours apart lead to a comparable luteolytic response to a single intramuscular injection of PGF, with similar P4 concentration achieved after treatment in cows. For an automated IVG delivery system, delivering two boluses of PGF will be as easy as delivering a single bolus—an example of how the programmability of the present technology enables adjustment or complicated protocols without adding labor or other cost.

### GnRH Studies

LH response of lactating Holstein cows after IVG delivery of GnRH was evaluated. Cows received IVG GnRH (Gonadorelin) at a standard dose of 100 μg, or at doses of 500 or 1,000 μg without and with citric acid as an absorption enhancer. A positive control group received 100 μg GnRH im and a negative control group received saline IVG. IVG GnRH induced an LH surge of similar magnitude to that induced by im injection as shown in FIG. 11. Adding 10% citric acid to the GnRH mixture (to promote absorption) was found to be necessary for efficacy of IVG GnRH, though a 10× greater GnRH dose was still needed than for im GnRH.

### Example 2—IVG Hormone Delivery Device

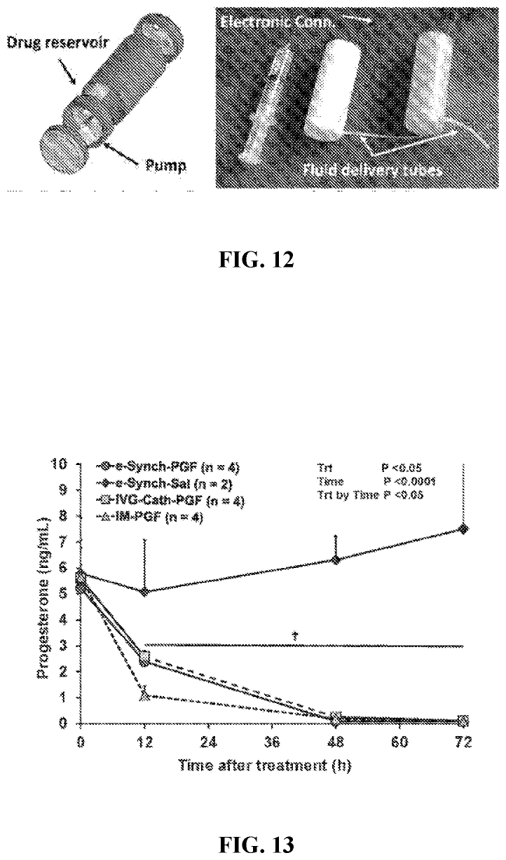

A single-chamber (single-hormone) prototype for IVG hormone delivery, as shown in FIG. 12, was formed. The prototype did not have integrated onboard electronics and power, but serves as a proof of concept and has allowed for testing of IVG hormone delivery from an in-situ reservoir (as opposed to catheter).

A proof-of-concept experiment was performed to demonstrate induction of luteal regression through electronically controlled IVG delivery of PGF. Lactating Holstein cows were randomized to receive two 50-mg doses of PGF 12 hours apart, or an equal amount of saline solution (negative control), or two 25-mg doses of PGF IVG (IVG positive control), or a single 25-mg dose of PGF im (im positive control). There were four cows in each group (except for saline, two cows). In cows treated with PGF via the prototype shown in FIG. 12, a pattern of P4 decline consistent with CL regression and similar to that of cows in the positive control groups was observed as shown in FIG. 13. In short, administration worked as well as standard injections. The device was easily inserted and removed and cows did not present any sign of discomfort before, during (˜12-14 min), or after treatments.

### Example 3—Electronically-Controlled Device for IVG Hormone Delivery

A fully automated intravaginal hormone-delivery device for reproductive control of cattle was developed. In-vitro and in-vivo validation work demonstrated that the current prototype device can be programmed to automatically release PGF2α for successful induction of luteal regression in lactating dairy cows. Once optimized, the developed intravaginal device may be an alternative tool to the needle-injection methods presently used to synchronize ovulation of cows. Future on-farm application of this automated system can potentially simplify herd management and reduce animal disruption.

The objective was to develop and validate an electronically-controlled hormone delivery device for reproductive control of cattle. After development and in-vitro testing of a prototype device for intravaginal (IVG) hormone release, the feasibility of inducing luteal regression by automated treatment with PGF2α (PGF) was demonstrated. The IVG device comprises an outer 3D-printed plastic housing, fluid reservoirs connected to delivery pumps and tubing, a programmable circuit board, and a retention mechanism. For in-vitro testing, 4 pumps were programmed to release different target volumes (0.1, 0.2, 0.5, 1.0, and 2.0 g) in 4 replicates (n=80). A Bland-Altman plot was constructed to assess the magnitude of disagreement between expected and delivered volumes. Observations fell within acceptable limits of agreement (1.96 SD) >95% of the time, indicating overall good agreement (mean difference=−0.005 g). To assess in-vivo performance of the IVG device, lactating Holstein cows with at least 1 corpus luteum ≥15 mm in diameter were randomly allocated to 1 of 3 treatments: IM-PGF (n=6); two 25 mg intramuscular doses of PGF (Dinoprost) 24 h apart, DEV-PGF (n=6); four 25 mg doses of PGF released automatically by the IVG device every 12 h, and DEV-CTL (n=4); insertion of an empty IVG device (placebo control). Blood samples were collected at 0, 12, 24, 36, 48, and 72 h after treatment. Data was analyzed by ANOVA with repeated measures in SAS. All devices (12/12) remained in situ until removed at 48-h. Progesterone (P4) concentrations from 0 to 72 h were affected by treatment, time, and their interaction. Concentrations of P4 did not differ at time 0 but differed from 24 to 72 h as cows in IM-PGF and DEV-PGF had lesser P4 than cows in DEV-CTL. Conversely, P4 did not differ for IM-PGF and DEV-PGF during the experiment. The current IVG hormone releasing device prototype can be programmed to automatically release PGF for successful induction of luteal regression in lactating dairy cows.

Timely and successful insemination of cows after they become eligible for pregnancy or a failed insemination is imperative to achieve optimal herd reproductive performance as described in Lamb, G., et al., “Control of the estrous cycle to improve fertility for fixed-time artificial insemination in beef cattle: a review.” J. Anim. Sci., 88:E181-E192 (2010), and Wiltbank, et al., “The cow as an induced ovulator: Timed AI after synchronization of ovulation.” Theriogenology, 81:170-185 (2014), the disclosures of which are incorporated herein by reference in their entirety. Therefore, a method used by many commercial farms to submit cows for insemination consists of synchronizing ovulation followed by timed AI (TAI) as described in Pursley, J., et al., “Synchronization of ovulation in dairy cows using PGF2a and GnRH.” Theriogenology, 44:915-923 (1995) and Pursley, J., et al., “Pregnancy rates per artificial insemination for cows and heifers inseminated at a synchronized ovulation or synchronized estrus.” J. Dairy Sci., 80:295-300 (1997), the disclosures of which are incorporated by reference herein in their entirety.

Benefits of TAI include insemination by appointment regardless of expression and detection of estrus and the possibility of achieving similar or greater fertility than by AI at detected estrus as disclosed in Pursley, J., et al., “Pregnancy rates per artificial insemination for cows and heifers inseminated at a synchronized ovulation or synchronized estrus.” J. Dairy Sci. 80:295-300 (1997) and Santos, V., et al., “Fertility of lactating Holstein cows submitted to a Double-Ovsynch protocol and timed artificial insemination versus artificial insemination after synchronization of estrus at a similar day in milk range.” J. Dairy Sci., 100:8507-8517 (2017), the disclosures of which are incorporated by reference herein in their entirety.

A major caveat of implementing TAI programs is the need to administer multiple hormonal treatments as intramuscular (IM) or subcutaneous injections. This problem is exacerbated as novel and more complex protocols are developed to maximize fertility, more cows need to be synchronized at the same time in larger herds, or for farms that lack critical resources to facilitate protocol implementation (e.g., dairy herd management software, proper facilities). Implementation of TAI protocols also requires significant human intervention and cow manipulation, which not only represent a cost burden for farms, but may also affect cow natural behaviors and time budgets as disclosed in Bolinger, D., et al., “The effects of restraint using self-locking stanchions on dairy cows in relation to behavior, feed intake, physiological parameters, health, and milk yield.” J. Dairy Sci. 80:2411-2417 (1997), the disclosure of which is incorporated by reference herein in its entirety.

Thus, a potential strategy to reduce the burden of implementing synchronization of ovulation protocols is to develop an all-encompassing delivery system for releasing all hormones of interest in the sequence, pattern, and dose required to synchronize ovulation. A requirement for successful synchronization of ovulation with an automated device is releasing hormones of interest at pre-defined time intervals at a rate and amount that elicit the desired physiological response. This is critical for reproductive hormones such as PGF_{2α} and GnRH which exert their biological effects (i.e., LH surge for GnRH and luteal regression for PGF_{2α}) by reaching target tissues in the form of sudden short-lived surges or pulses (i.e., minutes or a few hours) rather than in a sustained manner with elevated levels for prolonged periods of time (i.e., many hours or days). Another important consideration for the development of automated hormone delivery devices is placement within or on the cow body.

Among the different body part or cavities available for device placement, the vagina offers unique benefits. These include ease of insertion and removal, protection from damage or removal by contact with facilities or by other animals, constant temperature, suitability for extended retention, and efficacy of reproductive hormones after IVG delivery as disclosed in Wijma, R., et al., “Intravaginal instillation of gonadotropin-releasing hormone analogues with an absorption enhancer induced a surge of luteinizing hormone in lactating dairy cows.” J. Dairy Sci. 100:7626-7637 (2017), Wijma, R., et al., “Circulating progesterone dynamics after intravaginal instillation of prostaglandin-F2a to lactating dairy cows.” Theriogenology 85:16601668 (2016), and Masello, M., et al., “Intravaginal instillation of prostaglandin F2a was as effective as intramuscular injection for induction of luteal regression in lactating dairy cows.” J. Dairy Sci. 103:2743-2755 (2020), the disclosures of which are incorporated herein by reference in their entirety. Although previous efforts were made to develop some electronically controlled IVG hormone delivery devices (Cross, P. S., et al., “Control, communication and monitoring of intravaginal drug delivery in dairy cows.” Int. J. Pharm. 282:35-44 (2004) and Kunnemeyer, R., et al., “Electronically controlled, intravaginal drug delivery.” Proceedings of the Institution of Mechanical Engineers, Part B: Journal of Engineering Manufacture 218:1409-1415 (2004), the disclosures of which are incorporated herein by reference in their entirety), there is limited information about their performance and suitability for synchronization ovulation in cattle.

Therefore, a fully automated hormone delivery device for reproductive control of cattle was developed to conduct proof of concept in-vitro and in-vivo validation studies. For the in-vivo validation, we aimed to demonstrate complete luteal regression by automated delivery of PGF_{2α} because we have recently demonstrated similar luteal regression risk after IVG or IM administration of PGF_{2α} (as disclosed in Masello, M., et al., “Intravaginal instillation of prostaglandin F_{2α} was as effective as intramuscular injection for induction of luteal regression in lactating dairy cows.” J. Dairy Sci. 103:2743-2755 (2020), the disclosure of which is incorporated herein by reference in its entirety). It was hypothesized that automated delivery of PGF_{2α} by an electronically controlled IVG device would induce luteal regression and the changes in circulating P4 would be similar to those observed after IM injection of PGF_{2α}.

An IVG device included an outer 3D-printed plastic housing (12×4.0×3.0 cm), two silicone hormone reservoirs (˜5 mL) connected to delivery pumps (n=2; Takasgo Fluidic Systems, Westborough, Mass.), a printed circuit board (PCB) powered by a rechargeable battery, and a retention mechanism. The circuit board is programmed in C language to deliver target doses at a scheduled time. Two GPIO pins are routed and programmed to control the on/off switch of the n-channel MOSFETs (Diodes, Inc., Plano, Tex.), which controls the power supply for each peristaltic pump. In the “on” setting, liquid solution is pumped out of the hormone reservoirs through tubing that opens up to the exterior at the middle section of the device. Plastic elbows attached to each of the two orifices ensure proper liquid flow to the exterior of the device. To ensure ease of insertion and minimize irritation of the vaginal mucosa, the device is coated with skin-safe silicone rubber (Dragon Skin FX-pro, Smooth-on Inc., Macungie, Pa.).

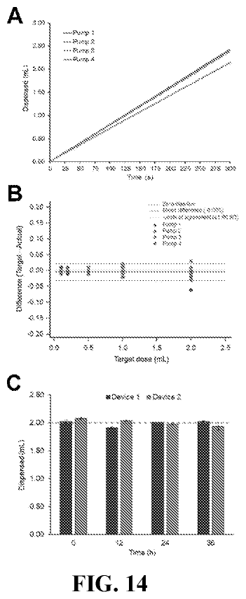

Once the final prototype was assembled, the delivery rate for each pump was determined by loading the hormone reservoirs with distilled water and measuring the amount delivered over a 300 s period using a precision scale (Radwag USA LLC., Miami, Fla.). Delivery curves for four different pumps are presented in FIG. 14A. Once the average release rate was defined, each pump (n=4) was programmed to release a total of 0.1, 0.2, 0.5, 1.0, and 2.0 g of water in four replicates (n=80 doses). In addition, long-term (i.e., 36 h) functionality was assessed in three replicates by programming IVG devices (n=2) to deliver four 2.0 g doses following a scheme of one dose every 12 h. The timing and duration of delivery (controlled by the on/off switch) was adjusted for each target dose based on the observed pump release rate (e.g., ˜250 s to deliver 2.0 g). To assess the magnitude of disagreement between the expected and delivered amounts and facilitate the detection of trends, a Bland-Altman plot (MedCalc, MedCalc Sofware bvba, Ostend, Belgium) was used to determine differences between target and delivered dose (y-axis) against target dose (x-axis). A minor bias across the range of target doses was observed (FIG. 14B). For doses in the 0.1 to 1.0 g range, all observations fell within the limits of agreement, whereas one observation from pump 1 (difference=−0.06 g, equivalent to 3% of target dose) and one observation from pump 2 (difference=0.03 g, equivalent to 1.5% of target dose) fell outside the limits of agreement for the 2.0 g dose. The overall difference between target and actual dose averaged −0.005 g, indicating overall good agreement. For the long-term assessment, both devices were able to accurately (<5% average error) release 2.0 g of distilled water every 12 h (FIG. 14C).

To assess IVG device performance in vivo, lactating nonpregnant primiparous and multiparous Holstein cows from the dairy unit of the Cornell University Ruminant Center (Harford, N.Y.) were enrolled in an experiment. Cows were housed in freestall barns with concrete flooring, self-locking head gates, and fans and sprinklers in the feedline. Cows were milked thrice daily at approximately 8-h intervals and were fed a TMR once a day with ad libitum access to feed and water.

All cows enrolled received a GnRH treatment (200 μg of gonadorelin acetate given intramuscular, Gonabreed, Parnell Pharmaceuticals, Overland Park, Kans., USA) at 40±3 DIM. Seven days later, transrectal ultrasonography (TUS) of the ovaries was performed using a 7.5-MHz linear probe (Ibex Pro; E. I. Medical Imagining, Loveland, Colo.). Cows with >1 corpus luteum (CL) ≥15 mm in diameter (n=16) were randomly allocated to 1 of 3 treatments: IM-PGF (n=6), DEV-PGF (n=6), and DEV-CTRL (n=4). Cows in IM-PGF received 2 treatments of 25 mg of PGF_{2α} (12.5 mg/mL of dinoprost tromethamine; Lutalyse HighCon, Zoetis, New York, N.Y.) 24 h apart as a 2 mL injection in the semimembranosus or semitendinosus muscle. Cows in DEV-PGF received 1 IVG device programmed to automatically release 4 doses of 25 mg of PGF_{2α} at ˜12 h intervals (first dose released at time 0). Cows in the DEV-CTRL treatment received an IVG device without PGF_{2α} to serve as a placebo control for the presence of the device in the vagina. All devices were removed at 48 h after insertion.

Before device insertion, the vulva and perineal area were washed and disinfected with chlorhexidine solution and dried off with paper towels. Thereafter, vulvar labia were manually opened by one technician while another technician inserted the device using a custom-built applicator. Before device insertion and after removal, a vaginal integrity score (0=no visible lesions, 1=superficial lesions, and 2=erosions of the vaginal mucosa; (Walsh et al., “Safety of a progesterone-releasing intravaginal device as assessed from vaginal mucosal integrity and indicators of systemic inflammation in postpartum dairy cows,” Can. J. Vet. Res. 72:43 (2008), the disclosure of which is incorporated herein by reference in its entirety) and a mucus score (0=clear mucus, 1=mucus with flecks of pus, 2=exudates containing <50% of pus, and 3=exudates containing ≥50% of pus; (Sheldon, “The postpartum uterus,” Veterinary Clinics: Food Animal Practice. 20:569-591 (2004), the disclosure of which is incorporated by reference herein in its entirety)) were determined for each cow using a vaginal speculum.

Blood samples (˜8 to 9 mL) were collected at time 0 and at 12, 24, 36, 48, and 72 h after treatment by puncture of caudal blood vessels using heparinized evacuated tubes (Vacutainer; BD, Franklin Lakes, N.J.). Samples were centrifuged at 2,000×g for 20 min at 4° C. Plasma aliquots were harvested and stored at −20° C. until assayed for progesterone (P4) in duplicate in 3 RIA assays performed as described in Beam, S. W., et al., “Energy balance and ovarian follicle development prior to the first ovulation postpartum in dairy cows receiving three levels of dietary fat.” Biol. Reprod. 56:133-142 (1997), the disclosure of which is incorporated herein by reference in its entirety. The average intra-assay coefficient of variation (CV) was 13.2% whereas the interassay CV was 17.0%. For this experiment, the presence of a functional CL was defined as circulating P4≥1 ng/mL (Ginther, O., et al., “Intrapulse temporality between pulses of a metabolite of prostaglandin F2a and circulating concentrations of progesterone before, during, and after spontaneous luteolysis in heifers.” Theriogenology 74:1179-1186 (2010), the disclosure of which is incorporated by reference herein in its entirety), whereas complete CL regression was defined as P4<1 ng/mL 72 h after treatment.

The effect of experimental treatments on P4 concentrations was evaluated by ANOVA with repeated measures using the MIXED procedure of SAS (version 9.4; SAS institute Inc., Cary, N.C.) with a model that included treatment, time, and the treatment x time interaction as fixed effects, whereas cow within treatment was included as a random effect. In addition, cow within treatment was the subject of repeated-measures analysis using a spatial power covariance structure to adjust for the varying time intervals at which blood was collected. Results presented are LSM±SEM. Significance was declared at P<0.05.

At the time of device insertion, all cows presented either clear or no vaginal mucus (mucus score=0), with no visible lesions of the vaginal mucosa (vaginal integrity score=0). At the time of device removal 4/10 cows presented mucus with flecks of pus (mucus score=1), and 5/10 cows presented mild irritation of the vaginal mucosa (vaginal integrity score=1). Irritation seemed to be located in areas where plastic elbows protruding from the device were in contact with the vaginal mucosa. Nevertheless, none of the cows had a score of 2 or erosions of the vaginal mucosa. In addition, none of the cows presented noticeable signs of distress or abnormalities in behavior at any time during the study period. All IVG devices (10/10) remained in situ for the 48-h study period (100% retention rate). Hormone reservoirs for all devices recovered from cows in the DEV-PGF group contained no fluid at the time of removal suggesting complete release while inserted.

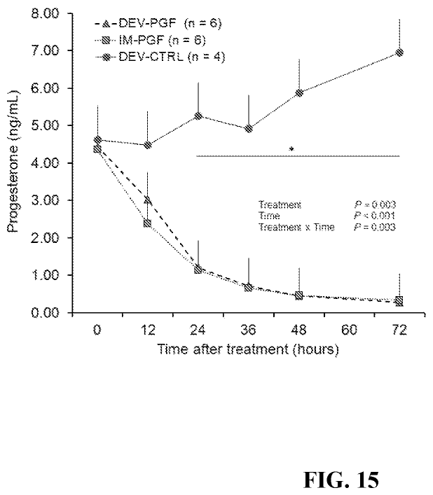

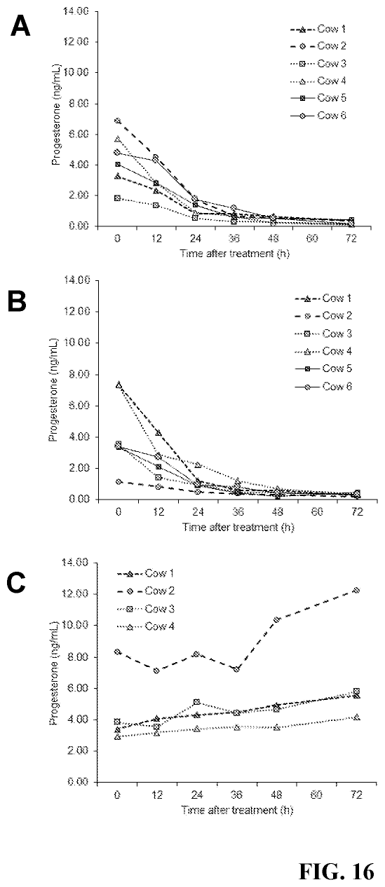

The effect of treatment on circulating P4 concentration profiles is presented in FIG. 15. There was an effect of treatment (P=0.003), time (P<0.001), and an interaction between treatment and time (P=0.003). From 24 to 72 h, cows in the DEV-PGF and IM-PGF treatments had lesser concentrations of P4 than cows in the DEV-CTRL treatment (negative control). In contrast, concentrations of P4 did not differ for the DEV-PGF and IM-PGF treatments during the entire sampling period. Circulating progesterone profiles for individual cows are presented in FIG. 16. Cows treated with PGF_{2α} had a 67 to 92% reduction in concentrations of P4 by 36 h after the first treatment. Except for one cow from the DEV-PGF (P4=1.20 ng/mL) and one cow from the IM-PGF treatment (P4=1.21 ng/mL), all other cows had P4<1 ng/mL at 36 h after treatment. Thereafter, concentrations of P4 continued to decline up to the end of the sampling period (0.14 to 0.50 ng/mL) when, except for one cow from the IM-PGF treatment (P4=0.50 ng/mL), all cows had P4 concentrations below <0.5 ng/mL. In contrast, cows in the DEV-CTRL treatment did not experience a decline in P4 concentrations at any point after device insertion and average P4 concentration for the group was never below 1 ng/mL.

A programmable, reusable IVG device for controlled hormone delivery in cattle was developed. The device is capable of automatically delivering up to two different types of hormones at predefined time points. Despite a minor bias observed with increasing amounts of target doses, in-vitro results showed that the current device permits precise control of released hormone amount and timing, and that performance does not seem to decrease overtime (at least for up to 36 h). This is relevant because causing the desired biological response with reproductive hormones such as PGF and GnRH depends on hormone release in the right amount at the right time with periods of several days in between treatments.

The device was able to automatically deliver PGF and induce complete luteal regression in lactating dairy cows. The differences in P4 profiles with the placebo controls and the lack of significant differences with IM-treated cows suggested that CL regression was caused by the PGF released by the device rather than the potential physical effect of the presence of the device in the vagina. These results were expected because we recently demonstrated similar P4 profiles and luteal regression risk in cows that received PGF through the IVG route or IM (Wijma, R., et al., “Circulating progesterone dynamics after intravaginal instillation of prostaglandin-F2a to lactating dairy cows.” Theriogenology 85:16601668 (2016) and Masello, M., et al., “Intravaginal instillation of prostaglandin F2a was as effective as intramuscular injection for induction of luteal regression in lactating dairy cows.” J Dairy Sci. 103:2743-2755 (2020), the disclosures of which are incorporated herein by reference in their entirety) and there is no biological reason to expect that the presence of the device in the vagina would cause CL regression.

A concern with the use of an electronic device for vaginal insertion is the potential detrimental effect of moisture and temperature on electronic components. In the current experiment, the device did not seem to be negatively affected by the vaginal environment as evidenced by the successful release of all the PGF loaded in the device reservoirs. This was likely because of the conformal silicone coating of the PCB and the extra protection provided by the external silicone rubber coating, which prevented moisture from affecting the device function. In addition, high retention rates and the long-lasting battery supply also contributed to the successful delivery of all doses of PGF. One of the limitations of the current experiment, however, was that the exact timing of PGF delivery could not be confirmed because the current version of the device was not designed to communicate with external sources. In-vivo monitoring of the timing of hormone release would have required device removal and re-insertion multiple times. This was avoided due to the potential irritation of the vaginal mucosa and cow discomfort due to repeated insertion and removal.

Mucopurulent vaginal discharge was observed in approximately half the cows that received a device. These findings were expected because it is known that insertion of IVG P4 releasing devices such as the PRID and CIDR-B might result in mucopurulent vaginal discharge in a substantial proportion of cows. The observed vaginal discharge, however, seemed to be inconsequential for fertility. In the current experiment, mild irritation of the vaginal mucosa on 40% of cows with an IVG device was also observed. This was likely caused by the plastic elbows protruding from the device because irritation was highly localized in small areas of the vaginal mucosa that may have been in contact with these elements of the device.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

What is claimed is:

1. A device comprising:
a housing configured to be inserted into a body cavity;
one or more reservoirs configured to be located within the housing and to store a fluid;
one or more pumps coupled to the one or more reservoirs, the one or more pumps configured to deliver fluid stored in the one or more reservoirs to an area external to the housing during use; and
a microcontroller coupled to the one or more pumps, the microcontroller configured to:
operate the one or more pumps to deliver a predetermined volume of the fluid stored in the one or more reservoirs to the area external to the housing at a scheduled time.

2. The device of claim 1, wherein the housing has a smooth outer surface to facilitate insertion into and removal from the body cavity.

3. The device of claim 1, wherein the housing further comprises a retention mechanism configured to retain the housing in the body cavity during use.

4. The device of claim 3, wherein the retention mechanism comprises a pair of opposing members configured to be folded into the housing and to provide an external pressure away from the housing against the body cavity during use.

5. The device of claim 1, wherein the housing further comprises an extraction mechanism to allow for removal of the housing from the body cavity.

6. The device of claim 5, wherein the extraction mechanism comprises a coil configured to extend from the housing outside of the body cavity during use.

7. The device of claim 1, wherein the one or more pumps coupled are to the one or more reservoirs by a mechanical coupling mechanism.

8. The device of claim 1, wherein the one or more pumps are negative pressure pumps.

9. The device of claim 1 further comprising:
delivery tubing coupled to the one or more pumps to deliver the fluid to the body cavity.

10. The device of claim 1, wherein the housing is configured to be inserted intravaginally.

11. The device of claim 1, wherein the microcontroller is configured to operate the one or more pumps to deliver the predetermined volume of the fluid stored in the one or more reservoirs based on a biological hormone administration schedule.

12. The device of claim 1 comprising:
a plurality of reservoirs, wherein each of the plurality of reservoirs are independently accessed by one of the one more pumps to allow independent fluid delivery from each of the plurality of reservoirs during use.

13. The device of claim 1, wherein the one or more pumps are configured to release the fluid from the one or more reservoirs in a surge release or a sustained release.

14. The device of claim 1, wherein the housing comprises a first component and a second component, wherein the first component houses the one or more pumps, the microcontroller, and a battery.

15. The device of claim 14, wherein the first component is sealed to prevent fluid from entering the first component.

16. The device of claim 14, wherein the second component is configured to be coupled to the first component.

17. The device of claim 14, wherein the second component is configured to removably receive the one or more reservoirs.

18. The device of claim 14, wherein the second component comprises a bladder configured to deform during operation of the one or more pumps to remove fluid from the one or more reservoirs.

19. The device of claim 1 further comprising:
one or more sensors coupled to the housing and the microcontroller, wherein the microcontroller is further configured to:
receive one or more items of data from the one or more sensors; and
adjust the operation of the one or more pumps based on the received one or more items of data to adjust the delivery of the fluid to the body cavity.

20. The device of claim 19, wherein the one or more items of data provide position information for one or more of the device or a subject in which the device is inserted, wherein the operation of the one or more pumps is adjusted based on the position of the device or the subject in which the device is inserted.

21. The device of claim 19, wherein the one or more items of data are based on physiological data.

22. The device of claim 21, wherein the operation of the one or more pumps is adjusted based on estrous behavior.

23. The device of claim 1, wherein the microcontroller is configured to receive information from an external device via a wireless communication network.

24. A method for providing automated fluid delivery in a body cavity, the method comprising:
providing the device of any one of claims 1 through 23;
inserting the device into the body cavity, wherein, upon insertion, the microcontroller operates the one or more pumps to deliver a predetermined volume of the fluid stored in the one or more reservoirs to the body cavity at the scheduled time.
